(19) United States
(12) Patent Application Publication
(10) Pub. No.: US 2022/0233229 A1
(43) Pub. Date: Jul. 28, 2022

(54) METHOD AND SYSTEM FOR CONTROLLING A SURGICAL HF GENERATOR, AND SOFTWARE PROGRAM PRODUCT

(71) Applicant: OLYMPUS WINTER & IBE GMBH, Hamburg (DE)

(72) Inventors: Thorsten JÜRGENS, Hamburg (DE); Andreas MÜCKNER, Schwarzenbek (DE); Per SUPPA, Hamburg (DE); Jakob MÜCHER, Hamburg (DE); Dennis BERNHARDT, Hamburg (DE); Andrea SCHWENDELE, Hamburg (DE); Veronika HANDRICK, Berlin (DE)

(73) Assignee: OLYMPUS WINTER & IBE GMBH, Hamburg (DE)

(21) Appl. No.: 17/581,378

(22) Filed: Jan. 21, 2022

(30) Foreign Application Priority Data
Jan. 22, 2021 (DE) 102021101410.7

(51) Int. Cl. A61B 18/12; A61B 5/02; A61B 5/00; G06V 20/52; G06V 10/82

(52) U.S. Cl. CPC A61B 18/1206; A61B 5/02042; A61B 2018/00589; G06V 20/52; G06V 10/82; A61B 5/7267

(57) ABSTRACT

A method and a system for controlling a surgical HF generator during a HF surgical procedure performed with a handheld HF surgical instrument supplied with HF energy by the HF generator. The method includes evaluating a succession of images of an operating area that are captured in an image sequence during the HF surgical procedure, including subjecting the images to automatic real-time image recognition to detect a predetermined structure and/or a predetermined operating situation, and in response to detection of the structure and/or operating situation, suggesting or performing a change of an operating parameter and/or operating mode of the HF generator. The system can include the HF generator, at least one handheld HF surgical instrument, a display device, a video endoscope, and a processor that is capable of receiving and evaluating image signals from the video endoscope.

### TECHNICAL FIELD

The present disclosure relates to a method, a system and a software program product having program code means for controlling a surgical high frequency (HF) generator during a HF surgical procedure with a handheld HF surgical instrument supplied with HF energy by the HF generator, wherein an operating area is monitored by means of image capturing, in particular by means of a video endoscope.

### BACKGROUND

Tissue in the interior of the body is treated during procedures using handheld HF surgical instruments. Examples of this are the closing of blood vessels by coagulation, the coagulation or cauterization of bleeding sites, or the cutting of tissue with simultaneous cauterization of the cut. In the case of a minimally invasive operation, in order to visualize the HF surgical procedure, two access points are usually made with two trocars, through which access points the handheld HF surgical instrument and an endoscope are introduced into the body cavity of the patient, for example the abdomen. The surgical field can be viewed directly during open surgery.

The various operative functions of handheld HF surgical instruments are produced by special HF waveforms. Since this involves an electromagnetic energy discharge, it is also possible to measure the treatment status of the treated tissue at the HF generator supplying the handheld HF surgical instrument with the various HF waveforms by way of the impedance of the treated tissue, which changes over the course of the treatment of the tissue. Thus, a blood vessel can be closed and coagulated, for example, with special HF waveforms, before it is dissected at the closure site. By utilizing special evaluation algorithms and monitoring the tissue response, it is possible to guarantee the safe coagulation of veins or arteries having a diameter of 5 mm, 7 mm or 9 mm, depending on the type of instrument deployed. It is then no longer necessary to utilize clips or stitches for the hemostasis.

HF energy is applied when the operator, commonly a surgeon, operates a corresponding operator control on the handheld HF surgical instrument. He is therefore in control at all times of how much HF energy is actually applied. In individual cases, this may be less than needed for achieving the desired success. To ensure that a coagulation is completed, HF generators are equipped, for example, with the function of generating an acoustic or audiovisual warning signal, a so-called “seal incomplete” sound, as long as a seal is not completely established. However, this causes irritation during operations if the surgeon does not apply the HF energy for hemostasis for the purpose of closing a blood vessel. In some cases, only small amounts of HF energy are needed, for example, to seal a small bleeding that occurred during a blunt dissection. However, the HF generator generates the signal tone in any case, resulting in unnecessary noise pollution and acoustic or audiovisual distraction in the operating theater.

HF generators are capable of providing HF energy available in a plurality of different HF waveforms and operating modes. Thus, monopolar and bipolar handheld HF surgical instruments can each be used with their own appropriate HF waveforms and operating modes which are suitable for the monopolar and bipolar instruments. A surgeon can select appropriate operating modes and HF waveforms from these, according to the current surgical situation. In the case of the applicant's HF generators, the surgeon can select a BiSoftCoag setting when using a bipolar handheld HF surgical instrument, for example for the preparation and mobilization of tissue, in which only slight bleedings are occurring. In the case of unexpectedly heavy bleedings, the HardCoag setting, by way of example, may be better suited. During an endoscopic mucosal resection (EMR) it may be advantageous to boost the setting of a monopolar electrode from SoftCoag to ForceCoag, or to treat a larger area with SprayCoag in order to stop a bleeding.

However, if an unexpected intraoperative situation occurs, valuable time may be lost if the surgeon is not able to set the HF mode correctly immediately.

### SUMMARY

The object which forms the basis of the present disclosure is to redress the disadvantages of the prior art.

This object can be achieved by a method for controlling a surgical HF generator during a HF surgical procedure performed with a handheld HF surgical instrument supplied with HF energy by the HF generator, in which an operating area is monitored by means of image capturing, such as by means of a video endoscope capturing a succession of images in an image sequence. The method includes evaluating the images including subjecting the captured images to automatic real-time image recognition to detect one or more predetermined structures and/or one or more predetermined operating situations, and, in response to the detection of one or more predetermined structures or operating situations, suggesting or performing a change of one or more operating parameters and/or operating modes of the HF generator.

The automatic detection of tissue structures and bleedings in captured images, in particular images acquired by endoscopes, by means of image analysis is state of the art. Common image recognition methods for detecting bleedings comprise, for example, color space transformations that can be used to increase the contrast between the color of openly escaping blood and the red shades of other tissue in the image.

One field of research in which this image analysis is being actively developed is the evaluation of images of capsule endoscopes. Capsule endoscopy (CE) is a non-invasive method for detecting abnormalities of, inter alia, the small intestine such as, e.g., bleedings. It provides a direct view of the patient's entire gastrointestinal tract. However, manual inspection of the huge number of images that are produced is tedious and lengthy, making it prone to human error. This makes automated computer-aided decision making attractive in this context.

In “Effective Deep Learning for Semantic Segmentation Based Bleeding Zone Detection in Capsule Endoscopy Images”, 25th IEEE International Conference on Image Processing (ICIP), pages 3034-3038 (2018), T. Ghosh, L. Li and J. Chakareski describe a deep learning-based semantic segmentation approach to detecting bleeding zones in capsule endoscopy images. Images of a bleeding have three regions which are characterized as bleeding, not bleeding and background. To this end, a convolutional neural network (CNN) having SegNet layers is trained with three classes. A given capsule endoscopy image is segmented with the aid of the training network and the detected bleeding zones are marked. The suggested network architecture is tested at different color levels and the best performance is achieved with the HSV color space (hue, saturation and value).

In L. Cui et al., “Bleeding detection in wireless capsule endoscopy images by support vector classifier”, Proceedings of the 2010 IEEE International Conference on Information and Automation, pages 1746-1751, an automatic algorithm for detecting bleedings in WCE images is suggested. This approach mainly focuses on color features, which are also a very effective indication used by doctors for diagnosis. Six color features in HSI color space are proposed for distinguishing between bleeding and the normal condition. A support vector classifier is used for checking the performance of the suggested features and for assessing the status of the images. The experimental results show that the proposed features and the classification method are effective and high accuracy can be achieved.

Image evaluation in capsule endoscopes is for an analytical purpose, since capsule endoscopes are only deployed for monitoring, not for the supervision of procedures. However, the images acquired by capsule endoscopes and by hand-operated endoscopes are very similar, so that the methods developed for capsule endoscopes can also be utilized for the images captured by video endoscopes and can be employed, in the context of HF surgery, to assist in the control of HF generators according to the present disclosure by suggesting or implementing changes of one or more operating parameters and/or operating modes of the HF generator. Depending on the type of the HF electrode of the handheld HF surgical instrument, this may be the selection of a monopolar or bipolar mode, for example one of the coagulation modes available for selection, or an operating parameter such as the HF voltage, HF waveform or the admission or suppression of an acoustic or audiovisual “seal incomplete” signal.

The method according to the present disclosure is not limited to the use of endoscopes. In open surgery, images from video cameras as image acquisition devices can also be used accordingly.

In embodiments, bleedings are detected as structures by means of the image recognition and a suitable HF mode for coagulation is suggested or applied as a change. For this purpose, in embodiments, a size and/or a blood volume of the bleeding is or are captured and is or are taken account of in the selection of the suitable HF mode.

In embodiments of the method, a semantic segmentation of the captured images is effected according to anatomical structures, in particular tissue types, organs and/or blood vessels. This provides a further improved context-sensitive decision basis for the selection of suitable HF modes and/or parameter changes. It can thus be taken into account that different organs and tissue types have different bleeding behavior. To this end, a detected bleeding can be attributed to the prevailing anatomical structure in said segment by its position in a segment of the image. During the selection of the suitable HF mode, a quality of the anatomical structure can be taken account of in addition to the size or intensity of a bleeding. Thus, a weak mode can be used, for example, to clamp or atrophy an arteriole, that is to say a small vein, whereas a spray coagulation is more suitable for incisions into the surface of an organ, for example the liver or a bile duct.

In embodiments, a bleeding is detected by means of an algorithm based on machine learning, in particular based on a neural network or a support vector machine, which has been trained with images or videos of organic structures with bleedings.

In a further development, a coagulation mode is suggested to an operator based on the detection of bleedings, and the algorithm is further trained with the current captured images based on feedback from the operator whether it is a bleeding or not, in particular based on the decision whether the detected site is treated by means of coagulation or not or whether a different mode or operating parameter to the suggested HF mode or operating parameter is used, wherein the further training is in particular carried out individually for various surgeons. This leads to an ongoing improvement in the detection reliability of bleedings and their context, and to the suggestions being increasingly appropriate to the situation. If the learning is individualized, the algorithm can adapt to the way different surgeons work.

In order to suppress disruptive and situationally inappropriate warning signals, the captured images are analyzed in embodiments of the method for a surgical situation in which a handheld HF surgical instrument is visible in the captured image, by means of which blood vessels can be sealed, and in which the handheld HF surgical instrument is approached to a blood vessel, wherein the handheld HF surgical instrument is identified from external data sources or from an image analysis designed for this purpose, wherein a probability is calculated that the approached blood vessel is to be sealed and an acoustic warning signal indicating an incomplete seal is suppressed if the probability lies below a predetermined or predeterminable threshold. To this end, in embodiments, the probability of whether the approached blood vessel is to be sealed is determined taking into account the progress of the approach, in particular a decreasing approach speed or a pause at the blood vessel, and/or taking account of the conditions of the approached blood vessel, in particular its skeletization, if applicable.

The detection of the operating situation and of the handheld HF surgical instrument can each likewise be trained. Thus, the analysis of the captured images for an operating situation can be based on a machine learning algorithm, in particular a trained neural network. The algorithm used for this purpose can, in a further development, be further trained with the current captured images based on the operator's decision whether or not to seal the blood vessel, wherein in particular the further training is carried out individually for different operators. If the learning is individualized, the algorithm can adapt individually to the way different surgeons work.

For this purpose, for example, the same neural network that had been trained to detect hemorrhage and, if applicable, semantic segmentation, may be or will be equipped with another classifier for handheld HF surgical devices. Alternatively, an independent algorithm can be deployed for the handheld HF surgical instrument. The type of the handheld HF surgical instrument can either be detected by means of the image recognition, or provided by the HF generator, if the HF generator and the handheld HF surgical instrument are equipped accordingly such that the HF generator automatically detects the type of the handheld HF surgical instrument.

The object underlying the present disclosure is also achieved by a system for controlling a surgical HF generator during a HF surgical procedure with a handheld HF surgical instrument supplied with HF energy by the HF generator, comprising the HF generator, at least one handheld HF surgical instrument that can be supplied with HF energy by the HF generator, a display device, a suggestion unit and an image evaluation unit, which is or are in particular part of the HF generator, and an image acquisition device, in particular a video endoscope signal-connected to the image evaluation unit, wherein the image evaluation unit is configured to receive and to evaluate image signals from the image acquisition device, characterized in that the suggestion unit and the image evaluation unit are configured and designed to perform a previously described method according to the present disclosure. The system realizes the same features, properties and advantages as the method according to the present disclosure.

Furthermore, the object underlying the disclosure is also achieved by a software program product with program code means with which a previously described method according to the disclosure is carried out, when the program code means relating to the image analysis are run in the image evaluation unit and the program code means relating to the control of the HF generator run in the suggestion unit, wherein in particular the image evaluation unit and the suggestion unit are configured as program units in a data processing device. The software program product also realizes the same features, properties and advantages as the method according to the disclosure.

Further features of the disclosure will become evident from the description of embodiments, together with the claims and the appended drawings. Embodiments according to the disclosure can fulfil individual features or a combination of multiple features.

Within the context of the disclosure, features which are labeled with “in particular” or “preferably” are to be understood to be optional features.

### BRIEF DESCRIPTION OF THE DRAWINGS

Exemplary embodiments will be described below without limiting the general concept of the disclosure by means of the exemplary embodiments with reference to the drawings, wherein reference is expressly made to the drawings regarding all of the details according to the disclosure which are not explained in greater detail in the text, wherein:

FIG. 1 shows a schematic representation of a system according to the disclosure for HF surgery,

FIG. 2 shows a schematic representation of a method according to the disclosure, and

FIG. 3 a schematic diagram of an exemplary computer-based clinical decision support system.

### DETAILED DESCRIPTION

In the drawings, the same or similar elements and/or parts are, in each case, provided with the same reference numerals such that they are not introduced again in each case.

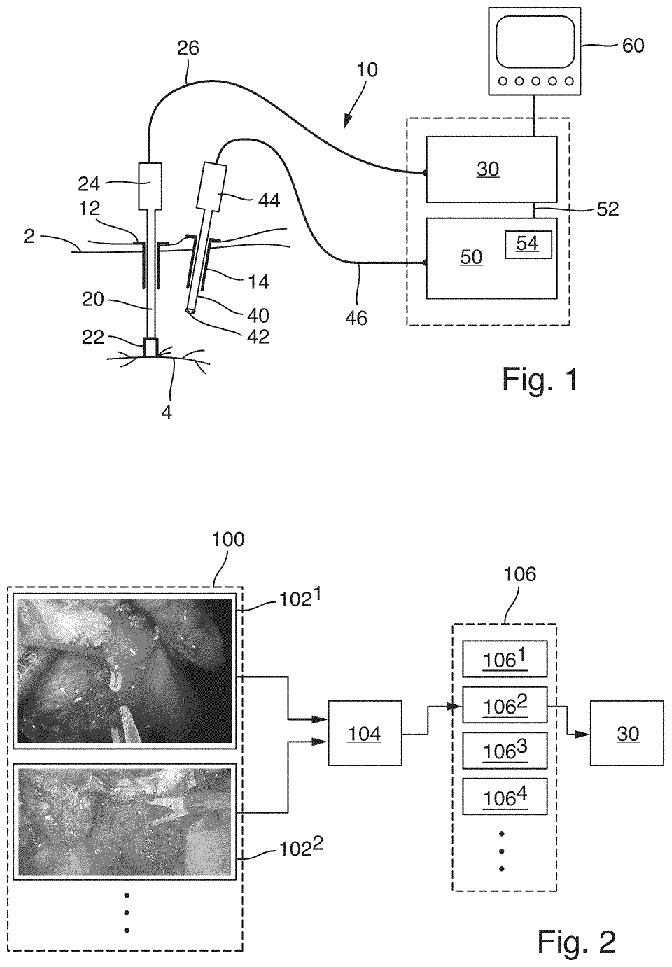

FIG. 1 shows a schematic representation of a system 10 according to the disclosure for use in HF surgery. A procedure with a handheld HF surgical instrument 20 and an endoscope 40 is performed in the abdomen of a patient. To this end, the handheld HF surgical instrument 20 and the endoscope 40 are introduced through two trocars 12, 14 through the abdominal wall 2 of the patient into the abdomen. The HF surgical procedures are performed with the handheld HF surgical instrument 20 having an HF electrode at its distal tip, which can be monopolar or bipolar, and a handle 24 at its proximal end outside of the abdomen, at which a surgeon holds and guides the handheld HF surgical instrument 20. In the representation of FIG. 1, the HF electrode 22 has engaged a blood vessel 4 to be sealed.

With its distal tip 42, the video endoscope 40 is oriented towards the blood vessel 4 and the HF electrode 22 of the handheld HF surgical instrument 20, so that both are located within the field of vision of the video endoscope. The video endoscope 40 can have its own light source (not represented) for illuminating the image field. The video endoscope 40 can be used in various embodiments. It can be of a type in which the image sensor is arranged in the distal part of the endoscope shaft behind a short inlet optic, or of a type that has a deflection optic up into the proximal part of the endoscope in the handle 44 where the image sensor is arranged. A further type consists of a conventional endoscope without its own image sensor and a camera head mounted on the endoscope which, together, form a video endoscope 40. Within the context of the present disclosure, the video endoscope 40 can be a stereo or a mono video endoscope.

The HF electrode 22 of the handheld HF surgical instrument 20 is connected via a supply cable 26 to a HF generator 30 that supplies the HF electrode 22 with HF energy. The HF generator 30 is configured to generate the HF energy in various HF modes with various waveforms, voltages, frequencies, etc. For this purpose, the surgeon can select between the various HF modes offered and, if applicable, additionally modify individual operating parameters of a HF mode in order to adapt these to the given operational requirements.

The video endoscope 40 is connected via a connecting cable 46 to an image evaluation unit 50, which receives and evaluates the image data of the video endoscope 40. The image data are, in addition, displayed in real time on a display device 60 of the system 10 in the field of view of the operating surgeon. The display device 60 is used to display both the image data from the video endoscope 40 and data regarding the selected HF mode and further operating parameters. The display device 60 may be a device having a touchscreen or having a conventional monitor and additional control knobs or panels in order to input changes to the HF mode or to operating parameters of the HF generator 30. Alternatively or additionally, the handheld HF surgical instrument 20 may also be equipped with operator controls such as push buttons, toggle switches or keys, thumb wheels or similar for confirming or rejecting as well as, if applicable, for selecting between various HF modes displayed on the display device 60 and/or operating parameters. Inputs via these operator controls are forwarded via the supply cable 26 to the HF generator 30.

The image data from the video endoscope 40 are evaluated in an image evaluation unit 50, wherein a suggestion unit 54 selects HF modes or parameters on the basis of the image evaluation and either transmits these directly, for example via a control line 52, to the HF generator or suggests them to the surgeon for selection via the display device 60. The image evaluation unit 50 and the suggestion unit 54 are either separate devices or are executed as software-implemented functional units within a data processing device. They can also be implemented in the HF generator 30.

An example of a method which can run in the system 10 is schematically represented in FIG. 2. An image sequence 100 with images 102¹, 102², . . . is acquired with the endoscope 40. By way of example, a photograph of an arterial bleeding is shown in image 102¹ and a photograph of a bleeding at the liver is shown in image 102². In addition, the HF electrodes of a handheld HF surgical instrument, which is executed as forceps, are shown in both images 102^{1,2}. The images are subjected to an automated structure and situation analysis in the image evaluation unit 50 in real time. Bleedings are detected as structures in the respective image 102^{1,2} and, if applicable, the size of the bleeding and/or the quantity of the escaped blood is/are captured.

In order to detect structures, a semantic segmentation can be effected according to anatomical structures. This would, for example, identify blood vessels as well as the bleeding in image 102¹ and, in addition to the bleeding, the surface of the liver as well as, if applicable, further tissue types in image 102². The bleeding can then be attributed, on the basis of its position in the respective image 102^{1,2}, to the blood vessel or the liver. During the selection of the suitable HF mode 106¹⁻⁴, a quality of the anatomical structure can be taken account of in addition to the size or intensity of a bleeding. It is therefore possible to select a suitable HF mode 106¹⁻⁴, in the suggestion unit 54, from a set 106 of preset HF modes of the HF generator 30, which is either made available to the operating surgeon for selection or is automatically implemented. The surgeon can be made aware of the change in the HF mode 106¹⁻⁴ or of another parameter acoustically or audiovisually, for example by a voice which announces the change and the adjusted HF mode 106¹⁻⁴.

The captured images 102^{1,2} can be analyzed for an operating situation 104. For example, there are handheld HF surgical instruments visible in the images 102^{1,2}. In the image 102¹, this could be a situation in which a blood vessel is to be sealed. This situation is very likely if the handheld HF surgical instrument 20 is brought closer still to a blood vessel 4. If the calculated probability that the blood vessel approached is to be sealed is low, then an acoustic or audiovisual warning signal which displays an incomplete sealing can be suppressed. This probability can be calculated, taking account of the progression of the approach, in particular a decreasing approach speed or pausing at the blood vessel. Conditions of the blood vessel approached can also be taken account of, in particular whether it is skeletized, which would be indicative of a high probability that the blood vessel is to be closed.

In another situation, it can be that, albeit a blood vessel is indeed detected in the image, a bleeding is also detected that cannot be attributed to that blood vessel. Then, a HF mode suitable for coagulating the existing bleeding is to be set or suggested initially. A “seal incomplete” warning may be omitted in such a case.

The handheld HF surgical instrument 20 can be identified from external data sources or from an image analysis designed for this purpose.

A bleeding can be detected by means of an algorithm based on machine learning, for example on the basis of a neural network or a support vector machine, which has been trained with images or videos of organic structures with bleedings such as the images 102^{1,2}. The same applies to the analysis of the captured images for an operating situation and for the decision regarding which HF mode 106¹⁻⁴ is to be suggested or which HF parameters should be amended. The learning algorithms can be further trained with the currently acquired images 102^{1,2}, for example on the basis of the decision of the operating surgeon as to whether the detected site is treated by means of coagulation or not, whether a different mode or operating parameter to the suggested HF mode 106¹⁻⁴ or operating parameter is used or whether a sealing of a blood vessel is effected or not. This continued learning may also be carried out specifically for individual surgeons so that the respective algorithm adapts itself automatically to the working characteristics of the respective surgeons.

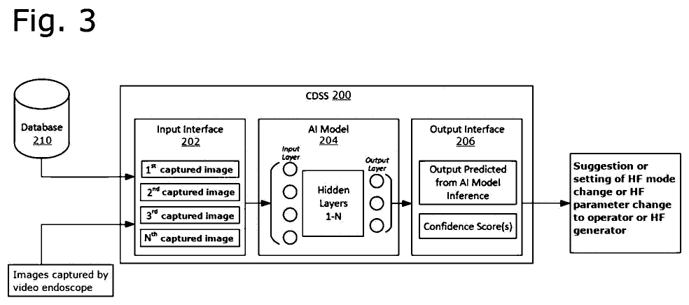

FIG. 3 shows a schematic diagram of an exemplary computer-based clinical decision support system (CDSS) 200 configured to output suggestions or settings for HF modes or parameters according to the present disclosure based on the results of the above-described image recognition.

The input to the CDSS are the images captured by a video endoscope as described above and may include training images from database 210. In various embodiments, the CDSS 200 includes an input interface 202 through which captured images from a surgical intervention are provided as input features to a processor 204 running an artificial intelligence (AI) model, which performs an inference operation in which the captured images are applied to the AI model to generate changed HF modes or parameters, and an output interface (UI) 206 through which the changes are communicated to a user, e.g., a clinician, for approval or denial, or directly to a HF generator.

In some embodiments, the input interface 202 may be a direct data link between the CDSS 200 and one or more medical devices that generate at least some of the input features. For example, the input interface 202 may transmit captured images directly to the CDSS during a therapeutic and/or diagnostic medical procedure. Additionally, or alternatively, the input interface 202 may be a classical user interface that facilitates interaction between a user and the CDSS 200. For example, the input interface 202 may facilitate a user interface through which the user may manually enter visible structures, operational situations or HF mode or parameter selections that may be used to further train the AI model 204. In any of these cases, the input interface 202 is configured to collect one or more of the following input features on or before a time at which the CDSS 200 is used to assess, whether a change in the selected HF mode or in currently applied HF parameters has to be carried out or suggested based on structures or operating situations found in the captured images.

Based on one or more of the above input features, the processor 204 performs an inference operation using the AI model to generate the above described system output. For example, input interface 202 may deliver the captured images and, if applicable, user inputs, into an input layer of the AI model which propagates these input features through the AI model to an output layer. The AI model can provide a computer system the ability to perform tasks, without explicitly being programmed, by making inferences based on patterns found in the analysis of the captured images. The AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model may be trained continuously or periodically prior to performance of the inference operation by the processor 204. Then, during the inference operation, the input features provided to the AI model, i.e., the captured images, may be propagated from an input layer, through one or more hidden layers, and ultimately to an output layer that corresponds to the system output, i.e. the changes to the HF modes and/or parameters to be implemented or suggested to the operator. Examples have been discussed above in relation to FIGS. 1 and 2.

During and/or subsequent to the inference operation, the change in HF modes or parameters may be communicated to the user via the user interface (UI) and/or automatically cause an HF generator connected to the processor 204 to perform a desired action. For example, the CDSS may inform a clinician of the patent specific AI generated output and prompt him or her to cancel or to confirm the suggested change in HF mode or HF parameter(s). Additionally or alternatively, the CDSS will cause the HF generator to perform the HF mode or parameter change immediately without consultation.

All of the indicated features, including those which are to be inferred from the drawings alone, and individual features which are disclosed in combination with other features, are deemed to be essential to the disclosure both alone and in combination. Embodiments according to the disclosure may be performed by individual features or a combination of multiple features.

### LIST OF REFERENCE NUMERALS

2 Abdominal wall
4 Blood vessel
10 System
12, 14 Trocar
20 handheld HF surgical instrument
22 HF electrode
24 Handle
26 Supply cable
30 HF generator
40 Video endoscope
42 Distal tip
44 Handle
46 Connecting cable
50 Image evaluation unit
52 Control line
54 Suggestion unit
60 Display device
100 Image sequence from the endoscope
102¹⁻² Images with bleeding situation
104 Structure and situation analysis
106 Set of preadjusted HF modes
106¹⁻⁴ HF mode
200 CDSS
202 Input Interface
204 Processor running AI model
206 Output Interface
210 Database

1. A method for controlling a surgical high frequency (HF) generator during a HF surgical procedure performed with a handheld HF surgical instrument supplied with HF energy by the HF generator, the method comprising:
evaluating a succession of images of an operating area that are captured in an image sequence during the HF surgical procedure, including subjecting the images to automatic real-time image recognition to detect a predetermined structure and/or a predetermined operating situation, and
in response to detection of the predetermined structure and/or the predetermined operating situation, suggesting or performing a change of at least one of an operating parameter and an operating mode of the HF generator.

2. The method according to claim 1, wherein a bleeding is detected as the predetermined structure by means of the image recognition and an operating mode of the HF generator that is suitable for coagulation is suggested or applied as the change.

3. The method according to claim 2, wherein a size and/or a blood volume of the bleeding is or are captured and the operating mode of the HF generator is selected based on the size and/or the blood volume of the bleeding.

4. The method according to claim 2, further comprising effecting a semantic segmentation of the captured images according to anatomical structures.

5. The method according to claim 4, wherein the anatomical structures include at least one of tissue types, organs, and blood vessels.

6. The method according to claim 3, further comprising effecting a semantic segmentation of the captured images according to anatomical structures,
wherein:
the detected bleeding is attributed to a prevailing anatomical structure in a segment based on a position of the detected bleeding in the segment of the image, and
the operating mode of the HF generator is selected based on a quality of the anatomical structure in addition to the size and/or blood volume of the bleeding.

7. The method according to claim 2, wherein the bleeding is detected by means of an algorithm based on machine learning.

8. The method according to claim 7, wherein the machine learning is a neural network or a support vector machine, which has been trained with images or videos of organic structures with bleedings.

9. The method according to claim 8, wherein:
a coagulation mode is suggested to an operator based on the detected bleeding, and the algorithm is further trained with current captured images based on feedback from the operator whether the detected bleeding is a bleeding or not.

10. The method according to claim 9, wherein the feedback from the operator whether the detected bleeding is a bleeding or not is determined based on whether a site of the detected bleeding is treated by the suggested coagulation mode or whether a different operating mode or operating parameter that is different from the suggested coagulation mode is used,

11. The method according to claim 8, wherein the further training is carried out individually for various operators.

12. The method according to claim 1, wherein
the captured images are analyzed for an operating situation in which the handheld HF surgical instrument is visible in a captured image, and in which the handheld HF surgical instrument is approaching a blood vessel that can be sealed by the handheld HF surgical instrument, and
the handheld HF surgical instrument is identified from external data sources or from an image analysis designed for this purpose, and
a probability is calculated that the blood vessel is to be sealed and an acoustic warning signal indicating an incomplete seal is suppressed if the probability lies below a predetermined or predeterminable threshold.

13. The method according to claim 12, wherein the probability of whether the blood vessel is to be sealed is determined by taking account of a progress of the approach, and/or taking account of the conditions of the blood vessel.

14. The method according to claim 13, wherein taking account of the progress of the approach includes taking account of a decreasing approach speed or a pause at the blood vessel, and taking account of the conditions of the blood vessel includes taking account of skeletization of the blood vessel, if applicable.

15. The method according to claim 12, wherein the captured images are analyzed for an operating situation on the basis of a machine learning algorithm including a trained neural network.

16. The method according to claim 15, wherein the algorithm is further trained with current captured images based on a decision by an operator whether or not to seal the blood vessel.

17. The method according to claim 16, wherein the further training is carried out individually for various operators.

18. The method according to claim 1, wherein the images of the operating area are captured by a video endoscope monitoring the operating area.

19. A system for controlling a surgical HF generator during a HF surgical procedure, the system comprising:
the HF generator,
at least one handheld HF surgical instrument that is configured to be supplied with HF energy by the HF generator,
a display device,
a video endoscope, and
a processor that is signal-connected to the video endoscope, and is configured to:
receive and to evaluate image signals from the video endoscope, and
perform the method according to claim 1.

20. A non-transitory computer readable storage medium having stored therein a program to be executable by a processor, the program causing the processor to execute:
evaluating a succession of images of an operating area that are captured in an image sequence during a HF surgical procedure, including subjecting the images to automatic real-time image recognition to detect a predetermined structure and/or a predetermined operating situation, and
in response to detection of the predetermined structure and/or the predetermined operating situation, suggesting or performing a change of at least one of an operating parameter and an operating mode of a HF generator.
